(19) United States
(12) Patent Application Publication
(10) Pub. No.: US 2022/0355265 A1
(43) Pub. Date: Nov. 10, 2022

(54) CONTROLLED PORE CERAMICS CHIPS FOR HIGH THROUGHPUT SOLID STATE OLIGONUCLEOTIDE SYNTHESIS

(71) Applicant: Nanopec, Inc., Tucson, AZ (US)

(72) Inventor: Mario Blanco, Tucson, AZ (US)

(73) Assignee: Nanopec, Inc., Tucson, AZ (US)

(21) Appl. No.: 17/736,134

(22) Filed: May 4, 2022

Related U.S. Application Data
(60) Provisional application No. 63/183,723, filed on May 4, 2021.

(51) Int. Cl. B01J 19/00; C07H 21/00; C40B 40/06; B82Y 30/00

(52) U.S. Cl. CPC B01J 19/0046; C07H 21/00; C40B 40/06; B82Y 30/00; B01J 2219/00722; B01J 2219/00596; B01J 2219/00585; B01J 2219/00659; B01J 2219/00725

(57) ABSTRACT

A nano-structured ceramic film with controlled pore size for the high throughput synthesis of oligonucleotides (DNA and RNA). The film can be cut into chips of predetermined size, and code printed for optical recognition in automated DNA synthesizers. The chips are easily activated under very mild conditions and silanization proceeds uniformly to allow reagents to flow unhindered through its open pores. Mono layer modifications, such as covalently bound silane coupling agents, allows for the addition of universal linkers and improved yields compared to conventional approaches.

### CROSS-REFERENCE TO RELATED APPLICATIONS

The present application claims priority to U.S. Provisional Application No. 63/183,723 filed on May 4, 2021.

### BACKGROUND OF THE INVENTION

### 1. Field of the Invention

The present invention relates to nanostructured ceramic films and, more specifically, to a nano-structured ceramic film with controlled pore size providing a chemically, optically and mechanically robust substrate for high-throughput solid state manufacturing of synthetic oligonucleotides.

### 2. Description of the Related Art

DNA synthesis occurs in all eukaryotes and prokaryotes. The molecular in vivo machinery involved in DNA synthesis has been studied extensively since the 1950's. The accurate synthesis of DNA is important in order to avoid mutations to DNA which could lead to diseases such as cancer. DNA biosynthesis in nature occurs via the polymerase chain reaction. In vivo DNA polymerases are highly accurate, with an intrinsic error rate of less than one mistake for every 10 million nucleotides added. In addition, some DNA polymerases also have proofreading ability; they can remove nucleotides from the end of a growing strand in order to correct mismatched bases.

Extracellular (in vitro) DNA amplification or gene synthesis—physically creating artificial gene sequences using the polymerase chain reaction and other enzymes in solution, is slow and prone to errors. Furthermore, for these to function a DNA template must first be constructed by other means. Purely chemical means to create DNA are now available that could one day have the potential to supersede in length and accuracy the in vitro biosynthesis methods without the need for a pre-existing template.

Fully synthetic oligonucleotides are critical in nearly all disciplines of modern biology and precision medicine. Synthetic Biology, cell-free DNA (cfDNA) diagnostics, mRNA vaccines, interference RNA cancer therapeutics, DNA Banking rely completely on the ability to generate error free oligonucleotide templates. Such revolution in biology has created a large demand for high-quality oligonucleotides, resulting in many companies developing novel approaches to improve DNA synthesis on solid substrates using Phosphoramidite chemistry. Controlled pore glass (CPG) columns lacks the quality necessary to create long sequences. Oligos produced from silicon chips are 2-4 orders of magnitude cheaper than column based CPG oligos, with costs ranging from $0.00001-0.001 per nucleotide, depending on length, scale and platform. This is due in great part to the broad particle size distribution which leads to incomplete synthesis of oligonucleotides trapped in small spaces (“shortmers”). In addition, CPG columns are too large and difficult to scale for the hundreds if not thousands of sequences needed to generate a full gene.

Silicon chips arrays have been developed to automate the accurate synthesis of thousands of oligonucleotides for gene assembly. One step coupling efficiencies of silicon chips have reached values on the order of 98.5% which makes them commercially feasible, however yields remain low. Because the amount of surface area for reactions on silicon chips have physical limitations yield of these chips is very low. Increasing the surface area with nano-lithography is possible but extremely expensive. Thus, a new solid substrate offering CPG class yields and the higher quality and automation capabilities of silicon chips is highly needed to make the promise of synthetic biology a reality.

This is a key bottleneck for high-throughput and inexpensive synthetic gene and genome construction. Solutions are needed with narrower pore size distributions as well as new formats beyond columns with a potential for miniaturization (chips), a necessary step for automation of large oligonucleotide synthesis. Here, we provide an nano-structured aluminum oxide ceramics film which when formed into bio-chips it provides a suitable solid state high-throughput substrate for oligonucleotide synthesis.

Any new substrate that intends to replace controlled pore glass (CPG) or silicon for oligonucleotide synthesis has to offer significant advantages, such as higher yields and better quality. It should also minimize the need for adjustments to existing DNA synthesis processes and standard operating procedures. It should also be chemically stable to solvents and reagents use in oligonucleotide synthesis. Finally, it should also offer optical and mechanical properties suitable for automation procedures such as split and pool, QR code optical micro-printing, and be offered in various sizes down to a few millimeters, or even micrometers, with stable crack free edges. Materials with these qualities cannot be made with inexpensive polymers which tend to swell in the presence of solvents. Most semiconductor materials require expensive nano-lithography to add sufficient surface area for high loading. This is a tall order since pricing should not exceed the costs of current nano-patterned silicon wafers. Accordingly, there is a need in the art for an improved substrate for oligonucleotide synthesis that can address these concerns.

### BRIEF SUMMARY OF THE INVENTION

The present invention comprises a nano-structured ceramic film suitable for the synthesis of oligonucleotides (DNA and RNA). The film can be cut into chips of various sizes, from a few centimeters to a few hundred microns without edge cracking. The surface of the film sufficiently optically uniform for code micro-printing commonly used in optical recognition by automated DNA synthesizers, such as in split and pool operations. The chips are easily activated under very mild conditions and silanization, the first step in chemically drafting an oligonucleotide, proceeds uniformly on a mono-layer, sufficiently thin to allow reagents to flow unhindered through its pores. Mono layer silanization, such as with APTES silane coupling agent, for addition of universal linkers have been demonstrated and when compared to CPG and silicon chips appear yields appear quite favorable.

Chips according to the present invention offered increased surface area over silicon (150× or higher) and extremely narrow pore size distribution (compared to CPG), with ease of activation, silanization and cleavage. Extensive chemical testing shows the chips are stable under solvents and reagents used in the most common Phosphoramidite oligonucleotide synthesis. The chips show significant higher oligonucleotide loadings than silicon chips which is the current standard for large scale DNA automation. These attributes provide a potential new material for the next generation of oligonucleotide chemistries with improve pore diffusion, eliminating ‘shortmers’, increasing the size of ‘error-free’ oligonucleotides when compared with CPG and increasing yields when compared to silicon chips. The ceramic films are manufactured without the use of heavy metals, such as mercury and chrome, commonly used in the manufactured of nano-porous ceramics. These characteristics bring these materials into the realm of potential cost-effective replacements for existing CPG columns and silicon chips for oligonucleotide synthesis automation.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a pair of scanning electron micrographs showing, in FIG. 1A, a nano-structured ceramic film according to the present invention having a high degree of order at the nanometer level, and in FIG. 1B, the microstructure of a commercially available controlled pore glass, SCHOTT Coralpore 1000, for comparison.

FIG. 2 is a pair of graphs illustrating the narrow pore size distribution for DNAReax obtained via digital processing of SEM images as compared to the pore size distribution of CPG-1000, showing the broad pore distribution for CPG compared to DNAReax.

FIG. 3 is a schematic of the various pore geometries that can be achieved through changes in synthesis parameters according to the present invention as each geometry may offer different advantages for loading and coupling efficiency to be optimized for specific oligonucleotide lengths and related constructs.

FIG. 4 shows, in FIG. 4A, a photograph of 2 cm×2 cm DNAReax bio-chips cut with rounded edges using a laser method that yields microcrack free edges and, in FIG. 2B, a 100× optical micrograph showing the ceramic crystal microstructure of DNAReax.

FIG. 5 shows, in FIG. 5A, a photograph of a group of smaller, 2 mm×2 mm, DNAReax metal composite chips where the composite consists of two external layers of DNAReax and a metal core layer in the middle to provide mechanical stability for oligonucleotide chemistry occurring under stirred solutions and mechanical sorting, such as is the case with the split and pool DNA synthesis method and, in FIG. 5B, a 10× micrograph of the side view of a composite DNAReax microchip showing the three layers.

FIG. 6 is a schematic showing DNAReax with pore size of 100 nm with two identical oligonucleotides representing a length of 100 nt depicted fully grown on both sides of the pore, where the space of the pore is sufficiently large to still permit the free flow of reagents.

FIG. 7 is a graph showing the chemical stability, percentage gain or loss over time, of DNAReax under common oligonucleotide solvents.

FIG. 8 is a graph is showing that DNAReax is also stable, with losses lower than 0.25% over a period of two hours, when immersed in ethylene diamine and diethylamine with large (10% by weight) catalytic amounts of water.

FIG. 9 is a table showing the rates of weight change and average changes for DNAReax samples for various oligonucleotide reagents, where the average absolute percent change is 0.23% across all reagents analyzed, and the maximum percent change, 1.15% over a period of 2 hours, is due to a mixture of 3% dichloroacetic acid in 97% dichloromethane, with all experiments carried out at room temperature under a hood.

FIG. 10 is a table showing that the average moisture ambient absorption of four samples of DNAReax is very low 0.034%, after the samples were activated, e.g., the surface has been hydroxylated in preparation for the addition of a universal linker, and room temperature was 20° C. and ambient moisture <10%. A monolayer of water is likely the source of this minimal moisture content.

FIG. 11 is an image showing the full spectral (emission vs excitation) spectrum of DNAReax after activation and derivatization with APTES, where strong emission at 520 nm confirms the presence of biotinylated fluorescein, which confirms the covalent bond between APTES on the surface and solution added avidin.

FIG. 12 is a series of fluorescence micrographs of DNAReax and glass samples after APTES surface modification where the uniformity and intensity of the fluorescent light indicates no segregation of APTES (A1, A4, A5, B3-B6, C1-C2), compared to glass (A2, A3, A6, B1, B2).

FIG. 13 is an image showing that the pores of DNAReax remain open after APTES modification and, more specifically, a drop of di-ionized water is able to penetrate the nano-porous film, without touching the edges, in less than one minute such that the structure remains open pore.

### DETAILED DESCRIPTION OF THE INVENTION

Referring to the drawings, wherein like numerals refer to like parts throughout, the present invention comprises a nano-structure ceramic material that can be used for the synthesis of long oligonucleotides. A ceramic film according to the present invention provides CPG class like loading with a much narrower pore size distribution, easier activation, and with optimal mechanical and optical properties comparable to silicon chips for oligonucleotide chemistry. In addition, the ceramic film of the present invention is highly stable in the most common solvents and reagents used in Phosphoramidite and related chemistries. Nano-structured aluminum oxide ceramics according to the present invention offer better controlled pore size distributions than conventional materials such as CPG, and are simpler to activate with a large reactive surface as compared to silicon chips. The present invention includes data that supports the robust nature of nano-structured aluminum oxide ceramics for carrying out Phosphoramidite chemistry, with minimal to no degradation under common solvents and reagents used for oligonucleotide synthesis. In addition, chips for high-throughput solid state oligonucleotide synthesis were manufactured and tested. Further properties were optimized for DNA automation, including optical quality suitable for character micro-printing and recognition. Metal/ceramic composites for mechanical strength were prepared for use in multiple split and pool operations where high sheer in solution and mechanical sorting can cause physical damage to an unsupported ceramic substrate.

Ceramic films according to the present invention are made through standard wet chemistry procedures without the use of heavy metals and without the need for costly nano-lithography. Due to its regular nano-pore structure, the ceramic film offers over a one hundred fold surface area for loading compared to plain or nano-structured silicon chips, while the pores are sufficiently wide to allow sufficient diffusion for synthesizing oligonucleotides above 300 bases long. For example, one embodiment of the present invention has a pore spacing that is regular, around 82 nm, with a pore diameter of about 60 nm. Thus, there are around 133 pores per square micron. However, these dimensions can be varied during fabrication to optimize specific oligonucleotide synthesis requirements of length and quality. The present invention can also be prepared as a composite of two ceramic films flanking a metal core to provide a mechanical strength that ceramic films alone lack. Throughout the present application, the nano-structured ceramic material of the present invention is referred to DNAReax for simplicity.

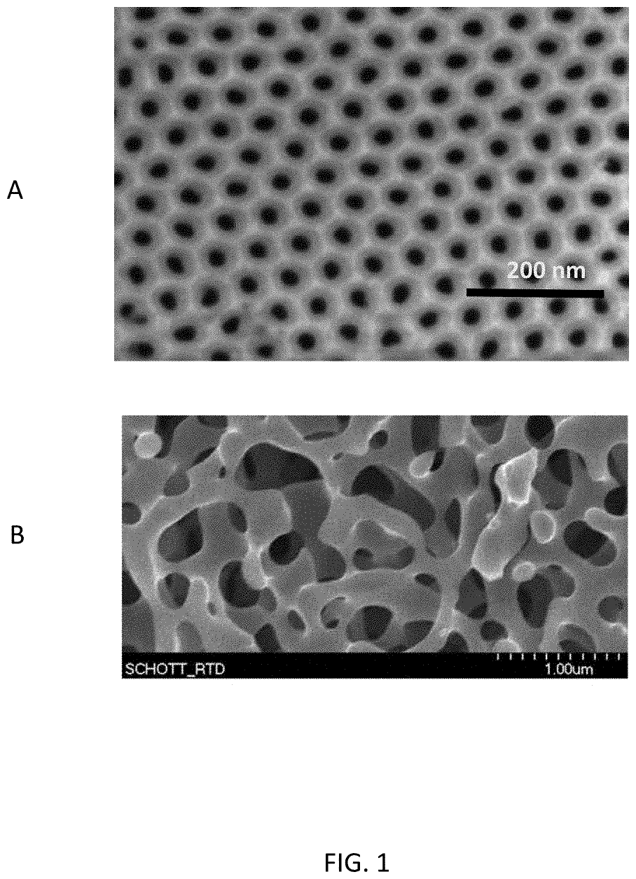

The ceramic films of the present invention employ the improved properties of anodic aluminum oxide, a class of nano-porous ceramic substrates that can be made in large scale and in good quality. FIG. 1A shows an example of a scanning electron micrograph of a nano-structured ceramic film for use as a robust substrate for high-throughput solid state manufacturing of synthetic oligonucleotides according to the present invention. The ceramic film can be manufactured as described in U.S. application Ser. No. 16/442,608, hereby incorporated by reference in its entirety. As seen in FIG. 1A, the ceramic film of the present invention shows a high degree of order at the nanometer level. For comparison the microstructure of a commercial controlled pore glass, SCHOTT Coralpore 1000, is shown in FIG. 1B.

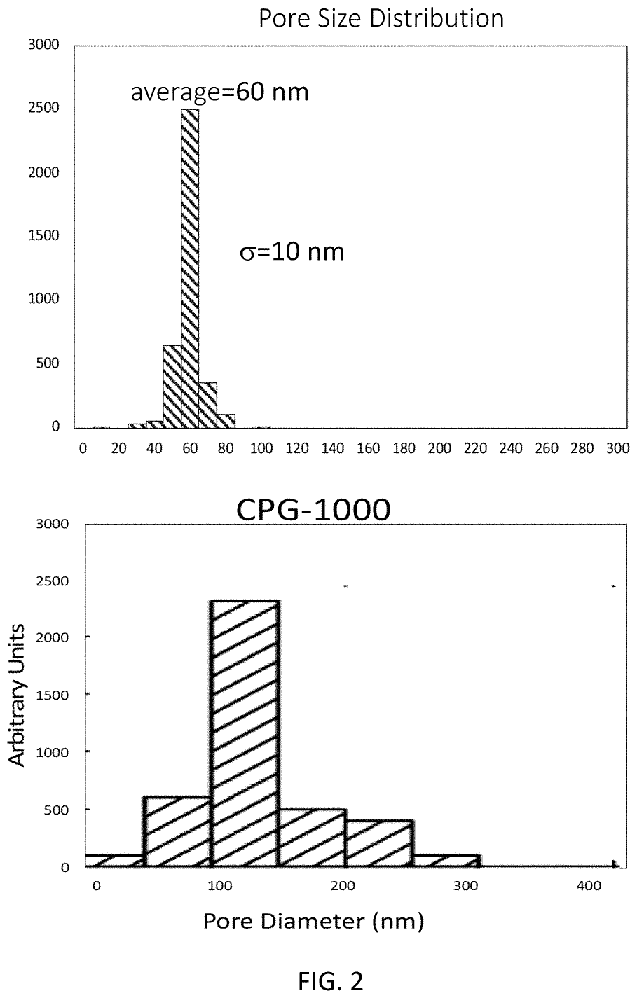

Referring to the first panel of FIG. 2, DNAReax has a narrow pore size distribution that can be determined through digital processing of SEM images and has an improved pore size distribution as compared to conventional materials, such as CPG-1000, a commercial brand of controlled pore glass, as seen in the lower panel of FIG. 2.

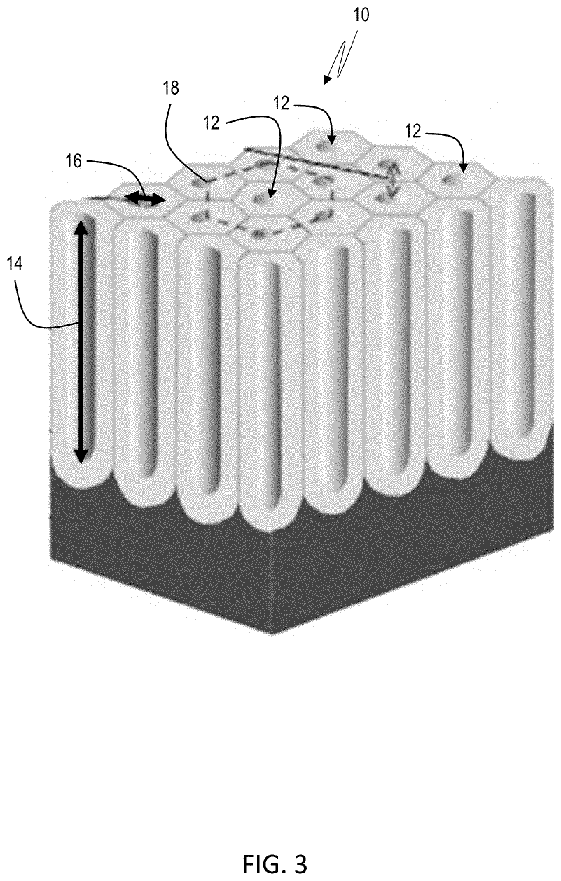

Referring to FIG. 3, various pore geometries can be achieved through changes in synthesis parameters of the present invention. Each geometry may offer different advantages for loading and coupling efficiency to be optimized for specific oligonucleotide lengths. For example, a substrate 10 according to the present invention may have a plurality of pores 12 each with a pore length 14 of up to 100,000 nanometers, a pore diameter 16 of up to 400 nanometers, and a lattice constant 18 of up to 500 nanometers.

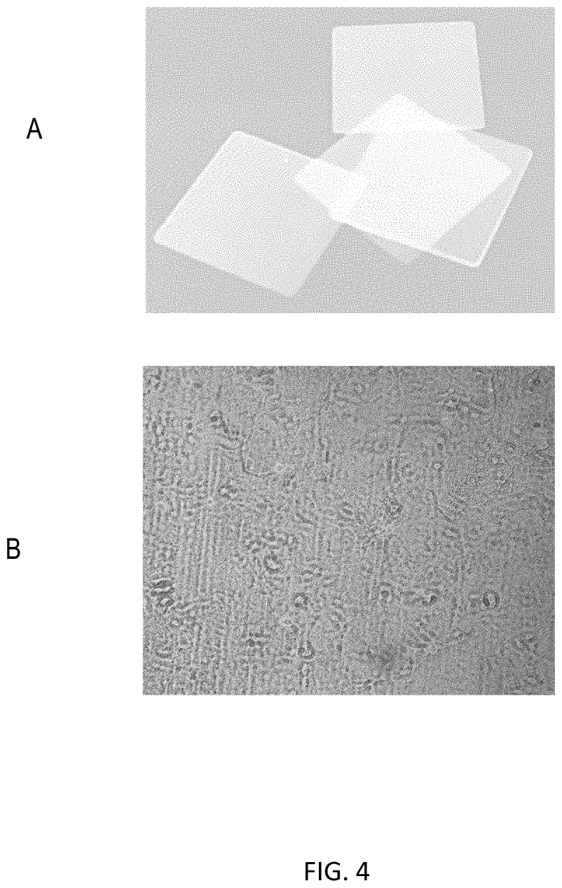

As seen in FIG. 4A, DNAReax according to the present invention can be configured as 2 cm×2 cm bio-chips cut with rounded edges using a laser method that yields microcrack free edges. FIG. 4B, which is a 100× micrograph of the surface of DNAReax, reflects the transparent crystallographic nature of the ceramic which does not interfere with microprinting, allowing the addition of soptical markings used for DNA synthesis flow processing.

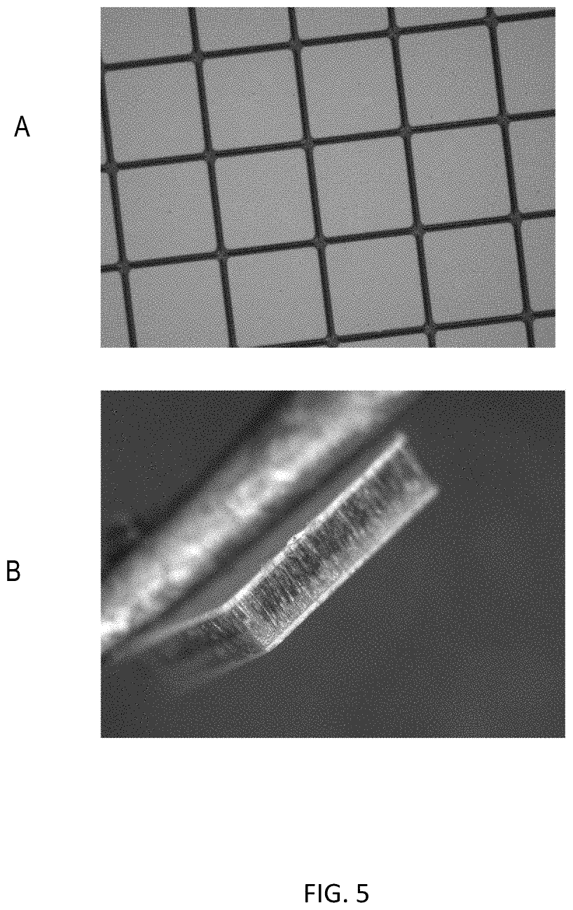

Referring to FIG. 5A, the present invention is configured as a group of smaller, 2 mm×2 mm, DNAReax metal composite chips. The composite consists of two external layers of DNAReax and a metal core in the middle to provide mechanical stability for oligonucleotide chemistry occurring under stirred solutions, such as is the case with the split and pool DNA synthesis method.

Referring to FIG. 5B, a composite DNAReax microchip has a central shiny layer that is pure metal and the two adjacent metal oxide layers are of similar thicknesses and porous at the nano-scale. A partial laser cut can be seen at the boundary between two neighboring chips. The edges are fused and no microcracks are present.

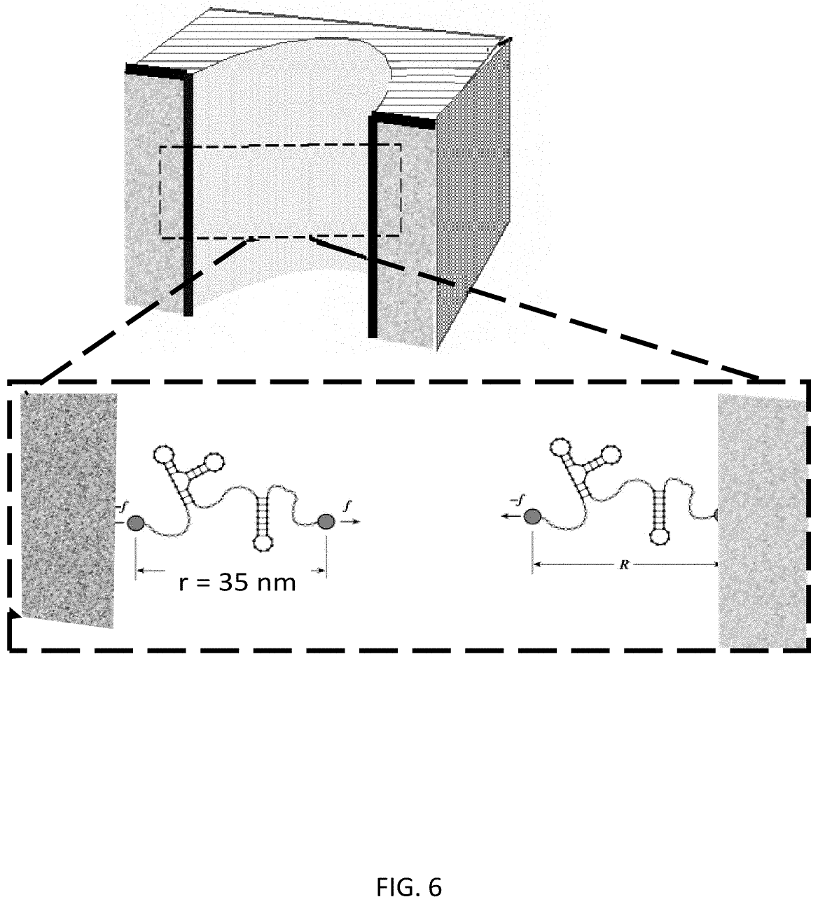

Referring to FIG. 6, DNAReax having a pore size of 100 nm can support two identical oligonucleotides representing a length of 100 nt depicted fully grown on both sides of the pore. In addition. the space of the pore is sufficiently large to permit the free flow of reagents. Pores between 50 nm and 400 nm in diameter are optimal to maximize synthesis yields. Ionic strength-dependent persistence lengths of single-stranded RNA and DNA have been measured accurately. The footprint (area) of a given sequence of ssDNA has been experimentally determined using volumetric analysis from atomic force microscopy (AFM) measurements. A simple estimate of the space inside these pores compared to accurate experimental radius of gyration of single strand DNA, ssDNA, sequences shows that such materials can sustain oligonucleotide synthesis on opposite sides of the pores up to 350 nucleotides. These are sizes beyond typically sequences synthesized in silicon chips (ca. 100 nt) and thus are more suitable for gene assembly with fewer strands.

Stability of DNAReax on Oligonucleotide Solvents and Reagent Exposure

The surface area of DNAReax can be as high as 150 to 400 fold, depending on thickness and pore size, compared to a piece of non-porous silicon film of the same planar dimensions. More surface area means higher reactivity, more sites to react or be dissolved. Therefore, quantitative data of the stability of DNAReax in the presence of solvents and chemicals used in Phosphoramidite chemistry was experimentally obtained using the following protocol.

A sample of DNAReax consisting of a square coupon 75 micron in thickness measuring approximately 2 cm×2 cm and weighing ca. 70 mg was weighed in a high precision balance (+/−10 mg). Before weighing the sample was oven dried for 1 hour at 102° C. and allowed to cool to room temperature.

Four samples were positioned in an hourglass and labeled by exposure time from 20, 40, 60 and 120 minutes. This is comparable to the exposure times for a 100 nt oligonucleotide synthesis procedure where each base addition cycle lasts from 30 to 60 seconds.

Each reagent was poured with a plastic disposable pipet inside a well-ventilated hood. Additional reagent was added as needed to keep the sample immersed, due to solvent evaporation.

After each exposure time the sample is lifted out of the hourglass and rinsed thoroughly with dehydrated ethanol followed by copious DI water.

After 1 min the sample is transferred to a non-fibrous paper to absorb excess water before transferring to a clean piece of roughened aluminum foil to avoid close contact with glass or the oven surface to avoid transfer of any material to the tested coupon.

Samples were dried in the oven for 1 hour at 102° C., subsequently cooled and weighed in a high precision microbalance, labeled and stored.

Because this was a static (no flow) test, the data in some cases may show a small (<2%) weight gain (precipitation) but generally a small loss (dissolution). When high humidity is present, the main dissolution product is aluminum hydroxide. Aluminum hydroxide eventually precipitates out as it is highly insoluble and may add weight to the sample. For specific cases, a 12 hour exposure was undertaken to test the limits of stability.

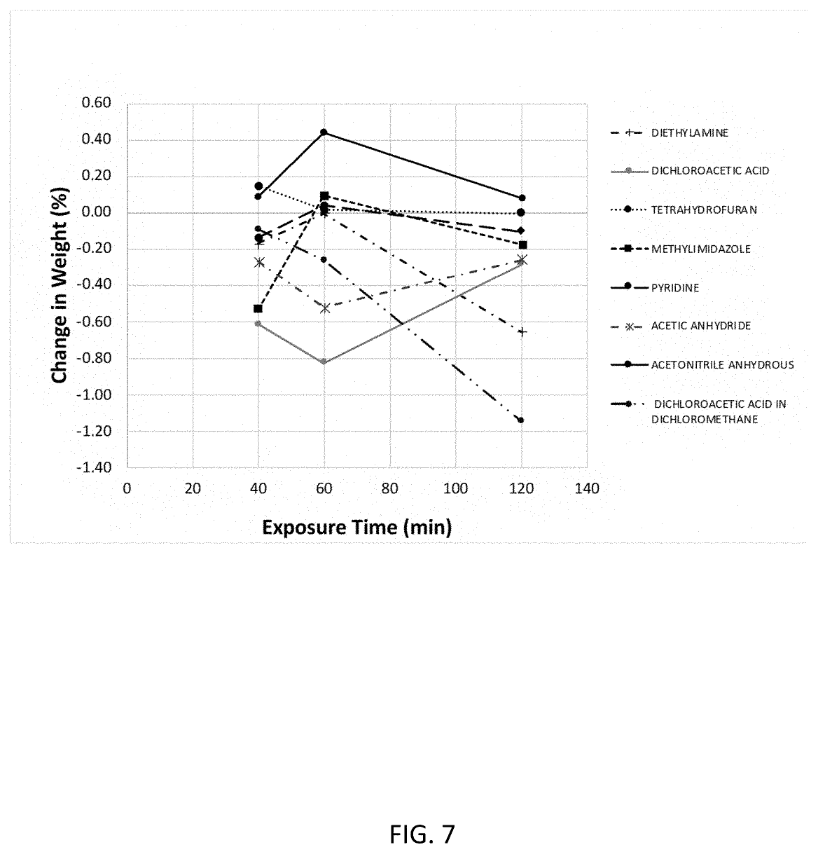

FIG. 7 summarizes the measures of the chemical stability of DNAReax under common oligonucleotide solvents. For exposures of up to one hour, the loss/gain is less than 1%. After two hours, the largest loss is less than 1.5% in dichloroacetic acid in dichloromethane. After two hours, a small gain can be observed in some cases but no more than 0.5% in all cases. This could be due to the presence of humidity in the as purchased solvents. These tests show the stability of DNAReax to the various solvents utilized in oligonucleotide chemistry.

Because aluminum oxide is sensitive to dissolution at high pH, a test was designed to test a strong base with small amounts of water. To furnish a functional oligonucleotide, all the protecting groups have to be removed. The N-acyl base protection and the 2-cyanoethyl phosphate protection are often removed simultaneously by treatment with inorganic bases or secondary or tertiary amines. Because water is often difficult to remove, its presence could alter the nature of the surface of aluminum oxide. Using an excess (10%) amount of water in the presence of two strong amines shows that DNAReax does not degrade over times significantly longer and at higher water content than required for deprotection.

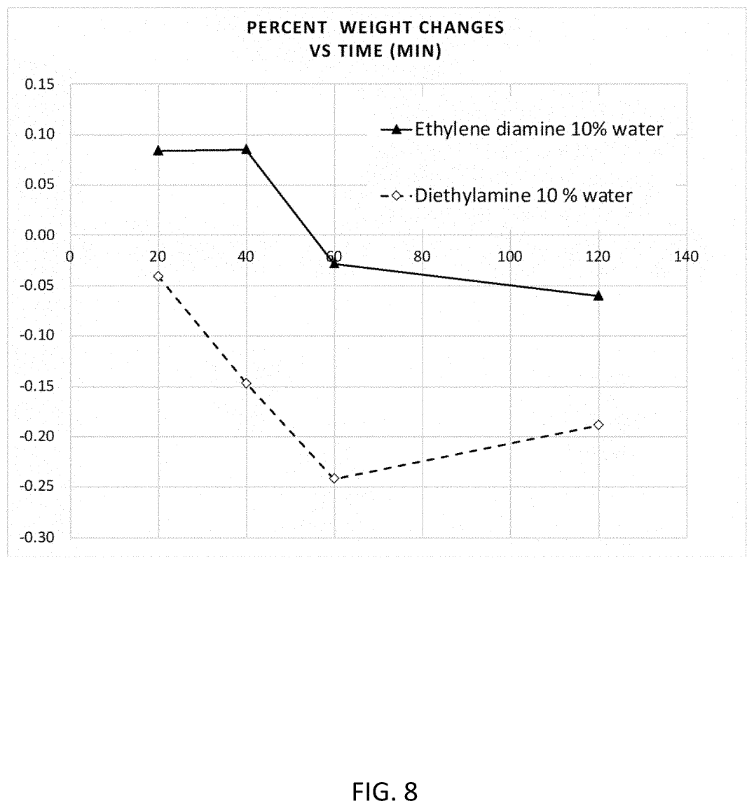

Referring to FIG. 8, DNAReax has been demonstrated to be stable, with losses lower then 0.25% over a period of two hours, when immersed in ethylene diamine and diethylamine in the presence of large (10% by weight) added amounts of water.

The list of reagents used in oligonucleotide chemistry is extensive. Furthermore, these are used often as mixtures. A representative list of reagents was chosen in close consultation with several research companies specialized in providing services to the oligonucleotide synthesis industry. The reagents are involved in all key steps of oligonucleotide chemistry. All reagents were purchased from Millipore Sigma Aldrich except for 3% dichloroacetic acid in dichloromethane, which was purchased from Glenn Research.

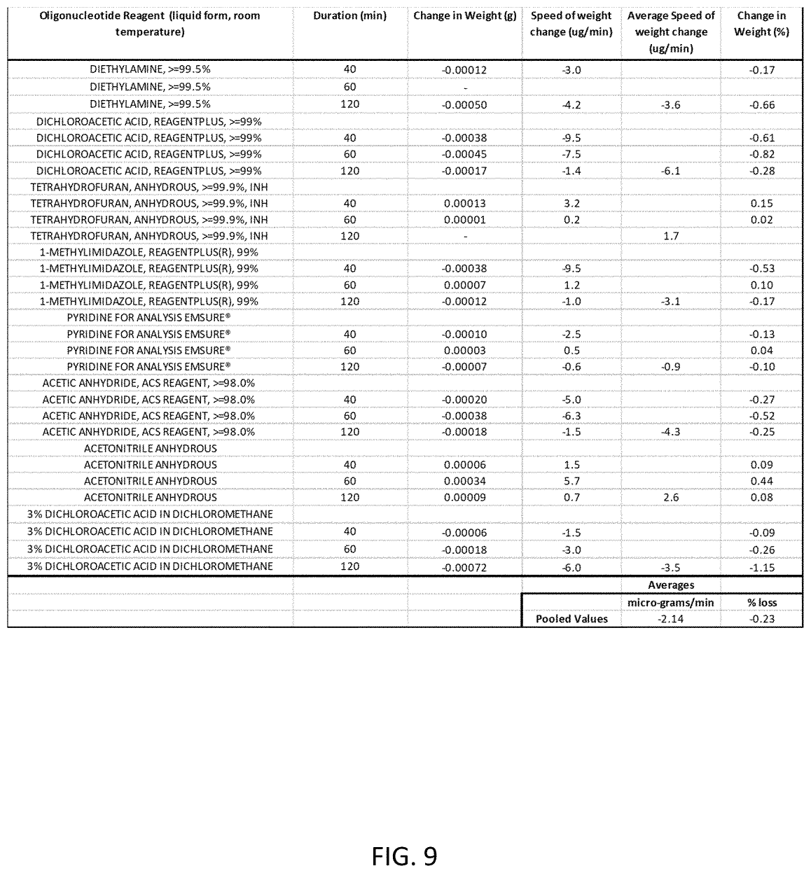

Referring to FIG. 9, the rates of weight change and average changes for DNAReax samples are shown for various oligonucleotide reagents. The average percent loss is 0.23% across all reagents analyzed. The maximum percent loss, 1.15% over a period of 2 hours, is due to a mixture of 3% dichloroacetic acid in 97% dichloromethane. All experiments carried out at room temperature under a hood as described in the protocol above. Moisture content is important since water can interfere significantly with coupling efficiencies. Four samples, approximately 0.07 grams in weight each, were weigh before and after oven drying.

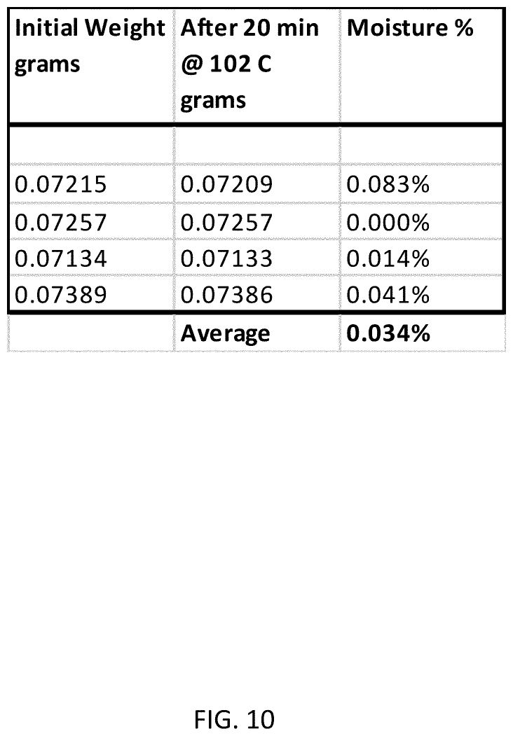

Referring to FIG. 10, the average moisture content of four samples of DNAReax according to the present invention was 0.034%. These samples have been activated, e.g., the surface has been hydroxylated in preparation for the addition of a universal linker. A monolayer of water is likely the source of this minimal moisture content. Room temperature was 20° C. and ambient moisture <10%.

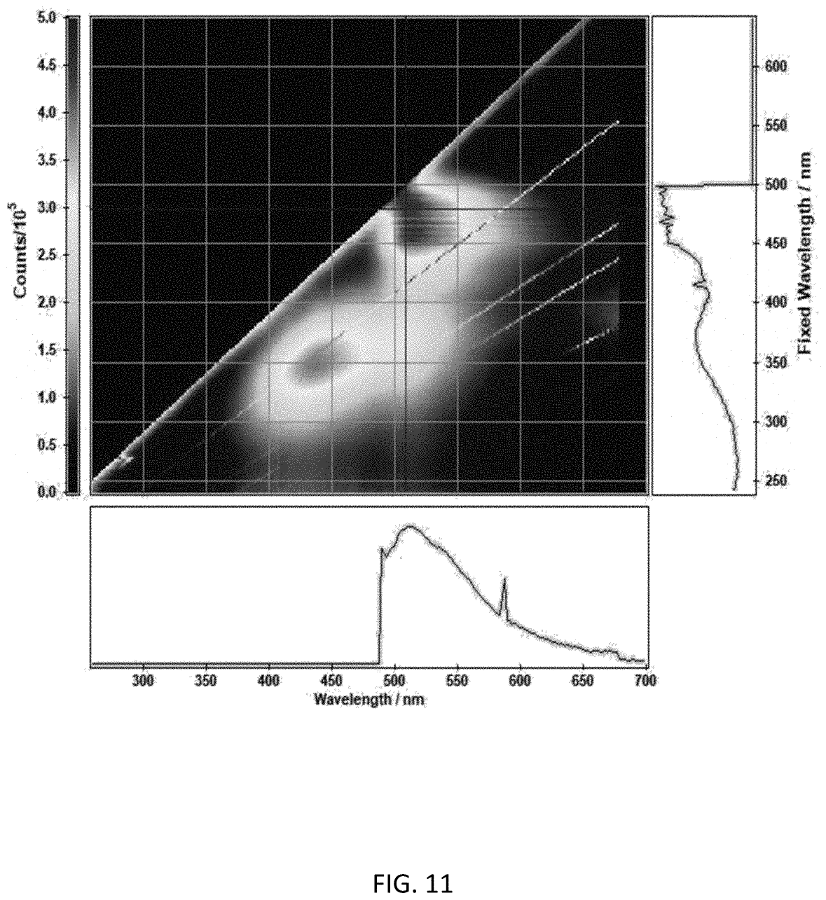

Referring to FIG. 11, the full spectral (emission vs excitation) spectrum of DNAReax after activation and derivatization with APTES may be seen. Strong emission at 520 nm confirms the presence of biotinylated fluorescein, which confirms the covalent bond between APTES and avidin.

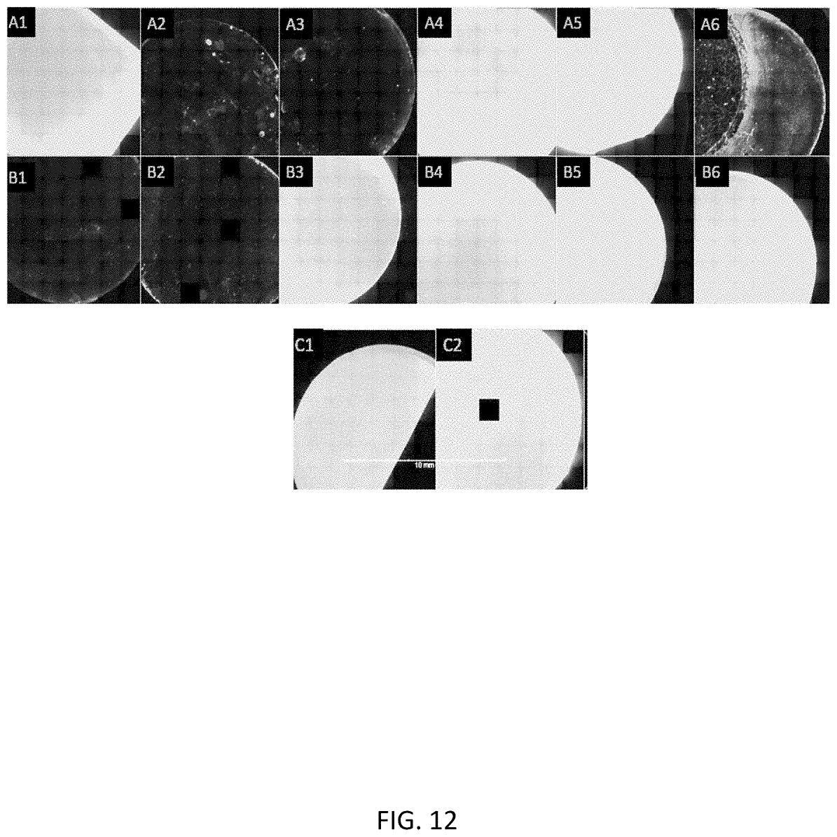

Referring to FIG. 12, fluorescence micrographs of DNAReax and glass after APTES surface modification demonstrate, via the uniformity of the fluorescent light, that there has been no APTES aggregation and that the coverage (intensity of fluorescence) is very uniform.

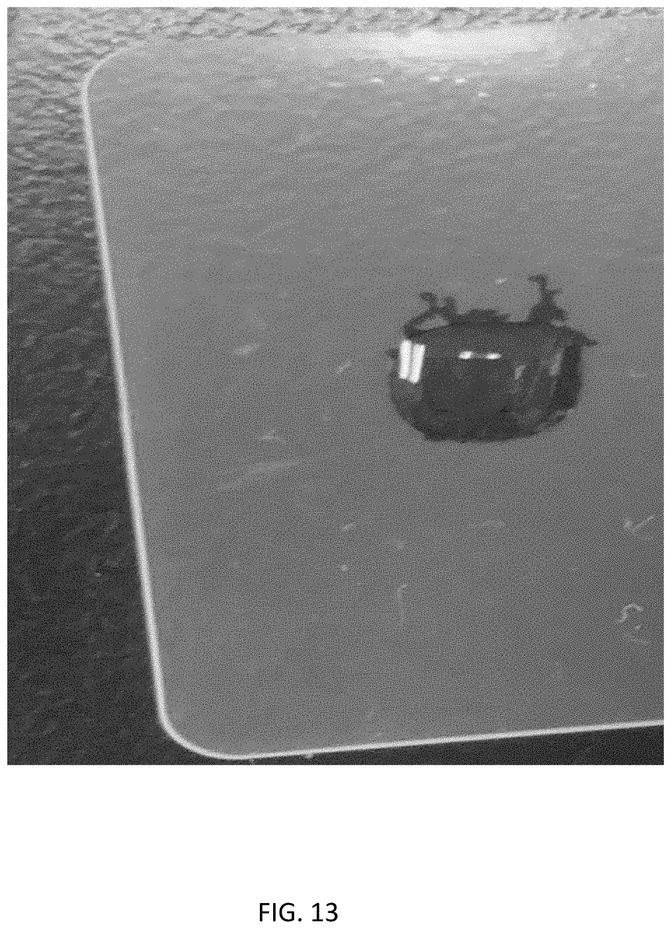

Referring to FIG. 13, the pores of DNAReax remain open after APTES modification. A drop of di-ionized water is able to penetrate the nano-porous film, without touching the edges of the chip, in less than one minute. The structure thus remains open pore.

Surface Modification for Oligonucleotide Synthesis

Prior to the start of the oligonucleotide synthesis, the solid support needs to be prepared with an organic compound that enables covalent attachment of a Universal Linker. A Universal Linker (UnyLinker) molecule is typically a chemically stable bridge carrying a conventional 4,4′-dimethoxytrityl (DMT) and succinyl groups to carry out oligonucleotide synthesis efficiently and smoothly. The organic compound is typically a silane coupling agent such as (3-Aminopropyl) triethoxysilane (APTES) or (3-Aminopropyl) trimethoxysilane (APTMS) that reacts with the metal oxide on one end and provides amino functionality to couple to the Universal Linker at the other end.

Activation

Prior to reacting the oxide surface with the silane coupling agent, the surface needs to be ‘activated’, i.e., the oxide surface groups transformed to hydroxide. For glass and silicon chips this usually requires strong acids such as nitric acid or even Piranha solutions. High temperatures are needed to hydroxylate the surface. DNAReax can be easily activated in 30% hydrogen peroxide at boiling temperatures (approximately 85° C.) for 15 minutes. After rinsing with sufficient ultra-pure water to eliminate any excess hydrogen peroxide DNAReax is dried for 2 hours at 102° C. in a convection oven and allowed to cool prior to applying the silane coupling agent.

Silanization: addition of a silane coupling agent, such as APTES, follows the typical published procedure used for silanization of CPG or silicon chips.

Results

The following protocol was used to check the uniformity of surface coverages with APTES.

Reagents:

Avidin (100 ug/mL in 1×PBS)

Phosphate Buffer Sulphate, PBS

Biotinylated-Fluorescein 50 uM

After APTES modification, various chips of DNAReax were processed as follows.

Derivatization with Avidin: DNAReax chips were incubated in sufficient avidin solution of 400 ul (100 ug/mL concentration in 1×PBS) for 2-3 hours at 37° C. in the dark, labeled and stored at 4° C. and covered with aluminum foil to protect them from light. DNAReax now contains immobilized avidin, chemically grafted with APTES. Avidin was chosen as a surrogate for a Universal Linker as it can be further imaged using a biotinylated fluorescent dye.

Confirmation of Silanization and derivatization with biotinylated fluorescein: this procedure verifies the APTES uniform effective coverage. Biotinylated fluorescein forms a strong non-covalent bond with Avidin in a 4:1 ratio

Prepare a known concentration of Fluorescence-4-biotin dye solution.

Recommended concentration is 50 mM.

Pour Fluorescence-4-biotin dye solution onto avidinated DNAReax with a pipet

Incubate for 2-4 hours at room temperature or overnight at 4° C. (covered with aluminum foil, protected from light).

Wash thoroughly 3 times with using TBS buffer pH=7.5 in sufficient amounts to rinse well all surfaces to eliminate any non-covalently bound fluorescein dye.

Quantification: to confirm the amount of fluorescein an Spectrofluorometer FS5 the full spectral signal (emission) was recorded for fluorescein, using 250 to 500 nm excitations. Note the high emission at 520 nm indicating the presence of fluorescein in FIG. 11.

Monolayer Coverage: For porous media such as DNAReax a simple method to verify that pores are not clogged with APTES, or any other modification, can be done with a water drop break through time test. A piece of APTES modified DNAReax was placed on a flat dark surface, such as the surface of a lab benchtop. A 50 microliter drop of ultra-pure water is placed on the surface and the time measured for the water to penetrate the ceramic chip and appear on the dark surface without reaching the edges, as in FIG. 13. The timer is stopped when the dendritic wetting of the surface beneath the sample is observed. Break through times can varied according to thickness of the ceramic film on the order of 10 seconds to 3 minutes for unmodified DNAReax. The test was run a second time by flipping the chip. For APTES modified DNAReax the breakthrough time is around 2-3 minutes for a 100 micron thick chip.

Uniformity of Silanization:

Uniformity of Coverage: fluorescent microscopy was used to verify the uniformity of coverage of the chip surface with a derivatized APTES surface. The results are compared to in FIG. 12. Note the absence of fluorescence concentration islands in DNAReax, which are present in APTES modified glass. Also notice the higher intensity of fluorescence in DNAReax chips compared to borosilicate glass (SCHOTT). A sodium glass gave similar results as SCHOTT despites claims that vintage sodium glass is more easily activated.

An Operetta CLS high-content analysis system with variable speed, sensitivity and resolution, fully-automated high-capacity, was used to image the surfaces. Sensitive sCMOS camera provided a large field of view and high resolution image capture. The Operetta's Harmony software was used to compare and contrast with similar procedure on 96-microwell with glass bottoms over a square grid.

Example 1: DNAReax samples are labeled: A1, A4, A5, B3, B4, B5, B6, C1 and C2. Illumination adjusted to the maximum 520 nm emission on the brightest (DNAReax) samples. Darker samples, A2, A3, A6, show incomplete or non-uniform APTES accumulation on SCHOTT and sodium glass samples, Bland B2, as evidence by the presence of bright spots (islands of fluorescein emission) over a mostly dark background.

Load Estimates

Three independent laboratories provided data to compare the oligonucleotide loadings on DNAReax with CPG and/or silicon chips

### Example 1

A major oligonucleotide independent laboratory provided loadings on DNAReax and compared them to CPG. Loadings of 200 nanomoles where achieved on a circular ceramic chip 20 mm in diameter. On a volume basis this is comparable to CPG but on a weight basis (DNAReax is 1.89 g/cc compared with CPG 0.4 g/cc) this is around 20% loading by weight.

### Example 2

A DNA synthesis laboratory conducted tests on columns packed with DNA chips. Two batches having two columns each were tested. The loads varied between 15.8 to 21.9 nanomoles/cm2 of DNAReax chip surface. On a weight basis this represents around 15% of the expected loads for their internal CPG column standards on a weight basis. Compared with state-of-the-art chip yields however this represent between 300% to 400% higher yields.

### Example 3

A different independent laboratory was asked to compare oligonucleotide loads on DNAReax with silicon chips used by the industry. An oligonucleotide with 30 bases was synthesized on DNAReax chips. Two separate batches of three chips each were tested. On average these batches gave yields between 4.9 and 8.3 nanomoles/cm2. These loads are 70 times higher than on silicon chips.

What is claimed is:

1. A substrate for synthesis of an oligonucleotide, comprising:
a ceramic film having a plurality of pores defining a plurality of interior pore surfaces;
wherein each of the plurality of pores has a diameter between 50 nanometers and 400 nanometers;
wherein the plurality of pores are spaced apart from each other a distance between 80 to 650 nanometers; and
wherein the plurality of interior pore surfaces are hydroxylated.

2. The substrate of claim 1, wherein the diameter of each pore is at least three times larger than a length of the oligonucleotide to be synthesized.

3. The substrate of claim 2, wherein the ceramic film has a thickness of between 50 and 150 microns.

4. The substrate of claim 3, wherein the ceramic film has sufficient optical uniformity for machine recognition of an optical code printed on the ceramic film.

5. The substrate of claim 4, wherein the ceramic film comprises a pair of layers surrounding and laminated to a non-porous core.

6. The substrate of claim 5, wherein the thickness of the non-porous core is sufficient to allow the pair of layers to withstand synthesis of the oligonucleotide.

7. The substrate of claim 6, wherein the non-porous core includes aluminum.

8. The substrate of claim 7, wherein the substrate is formed into a biochip having rounded edges for use in an automated synthesizer.

9. The substrate of claim 8, wherein the substrate includes a monolayer of a silane coupling agent suitable for oligonucleotide synthesis.

10. The substrate of claim 3, wherein the substrate includes a monolayer of a silane coupling agent suitable for oligonucleotide synthesis

11. The substrate of claim 9, wherein the surface is modified with a universal linker molecule.

12. The substrate of claim 10, wherein the pores of the substrate are open at one end to allow diffusion of any reagent used in the synthesis of the oligonucleotide.

13. A method of synthesizing an oligonucleotide, comprising the steps of:
selecting a ceramic film having a plurality of pores defining a plurality of interior pore surfaces that are hydroxylated, wherein each of the pores has an inner diameter that is at least three times a length of the oligonucleotide to be synthesized; and
modifying the interior pore surfaces to attach a universal linker; and
synthesizing the oligonucleotide one nucleotide at time beginning at the universal linker.

14. The method of claim 13, wherein the step of modifying the interior pore surfaces includes the step of attaching a silane coupling agent.
